(19) United States
(12) Plant Patent Application Publication
(10) Pub. No.: US 2015/0067933 P1
(43) Pub. Date: Mar. 5, 2015

(54) Cherry rootstock plant named 'STO2'

(50) ;

(71) Applicant: Peter Stoppel Obstbau, (US)

(72) Inventor: Peter Stoppel, Kressbronn (DE)

(21) Appl. No.: 13/987,742

(22) Filed: Aug. 27, 2013

(51) Int. Cl. A01H 5/00

(52) U.S. Cl. PLT/183

(57) ABSTRACT

A new and distinct cultivar of Prunus plant named ‘STO2,’ characterized by its weeping plant habit and typical weak growth; good adaptation at hot and dry conditions; and as a rootstock, weak vegetative growth that imparts uniformity, higher yield and better fertility to the grafted variety.

### LATIN NAME OF THE GENUS AND SPECIES

The Latin name of the genus and species of the plant variety disclosed herein is Prunus cerasus L.×schmittii Rehder.

### VARIETY DENOMINATION

The inventive cultivar of Prunus cerasus L.×schmittii Rehder disclosed herein has been given the varietal denomination ‘STO2.’

### BACKGROUND OF THE INVENTION

The present invention relates to a new and distinct cultivar of Prunus plant, typically used as a cherry tree rootstock, botanically known as Prunus cerasus L.×schmittii Rehder, and hereinafter referred to by the name ‘STO2.’

The new and distinct variety of cherry hybrid tree of the present invention was bred as a cross of Prunus cerasus with a Prunus canescens hybrid in Germany in 1989. It has been successfully asexually propagated by tissue culture. The purpose of breeding program was to find a better adapted cherry rootstock. The new Prunus plant is better adapted on heat and aridity than other rootstocks with a weak vegetative growth. The breeder also succeeded in creating a new cherry rootstock, which has a higher yield and influences the grafted variety positively in fertility.

### SUMMARY OF THE INVENTION

Plants of the new Prunus have not been observed under all possible environmental conditions. The phenotype may vary slightly with variations in environment such as temperature and light intensity without any variance in genotype.

The following traits have been repeatedly observed and are determined to be the unique characteristics of ‘STO2.’ These characteristics in combination distinguish ‘STO2,’ as a new and distinct cultivar of Prunus:

1. Weeping plant habit;

2. Good adaptation to hot and dry clime conditions;

3. As a rootstock, with weak vegetative growth, it produces a dwarf tree;

4. As a rootstock, imparts uniformity and higher yield to the grafted variety;

5. As a rootstock, trees are around 70% weaker than on Prunus avium seedlings; and.

6. Absence of suckers.

Plants of the new Prunus can be compared to plants of Prunus ‘GI 2091’ (U.S. Plant Pat. No. 16,173, issued Dec. 27, 2005). In side-by-side comparisons conducted in Kressbronn, Germany, plants of the new Prunus differed primarily from plants of ‘GI 2091’ in the following characteristics:

1. As a rootstock, plants of the new Prunus have about 10% stronger growth in comparison to plants with ‘GI 2091’ rootstocks;

2. Plants of the new Prunus do not develop stipules, whereas ‘GI 2091’ plants develops stipules;

3. Plants of the new Prunus have a weeping habit, whereas ‘GI 2091’ plants have an upright habit; and

4. Due to its typical/characteristic growth, plants of the new Prunus impart higher yield to the scion plant than plants of ‘GI 2091.’

Asexual reproduction of Prunus rootstock plant ‘STO2’ by tissue culture since 2001 has shown that the unique features of this new variety are stable and the plant reproduces true to type in succeeding generations of asexual reproduction.

### BRIEF DESCRIPTION OF THE DRAWINGS

The accompanying photographs illustrate the overall appearance of the new Prunus plant. These photographs show the colors as true as it is reasonably possible to obtain in colored reproductions of this type. Colors in the photographs may differ slightly from the color values cited in the detailed botanical description which accurately describe the colors of the new Prunus plant.

FIG. 1 provides a side perspective view of a typical tree of ‘STO2’ grown in an outdoor orchard.

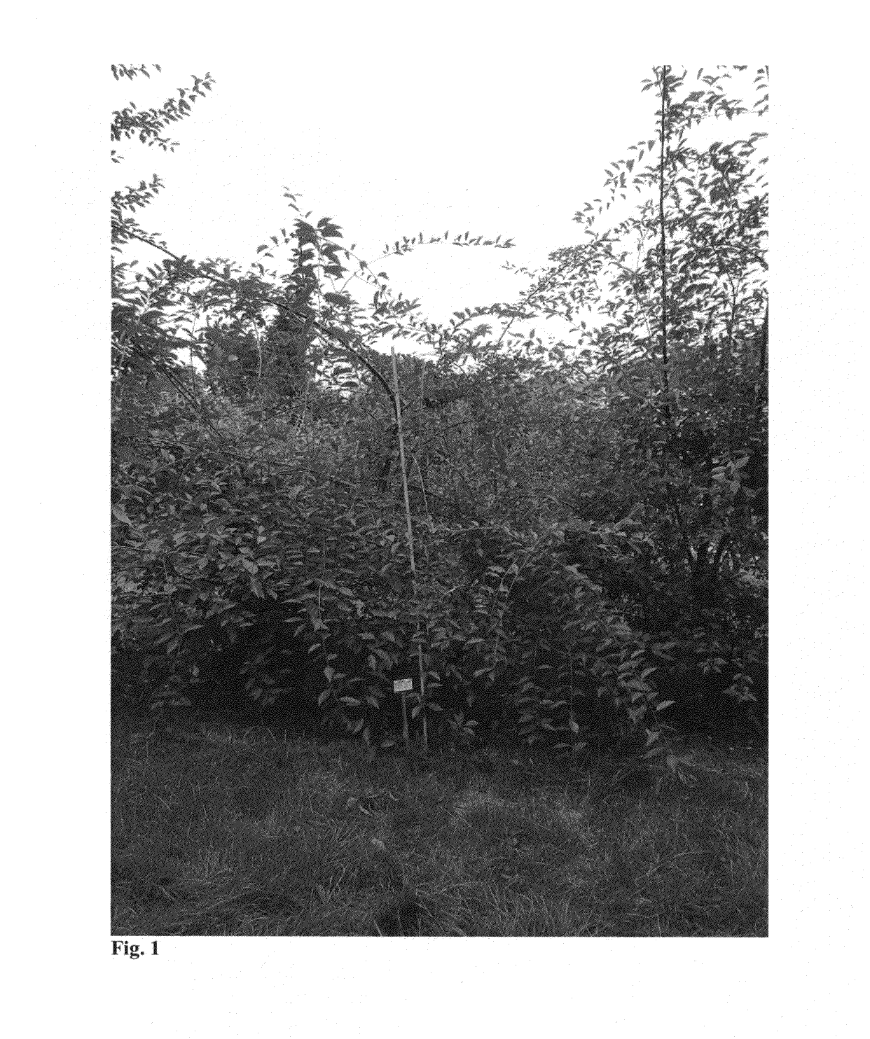

FIG. 2 provides a close-up view of leaves of a tree of ‘STO2.’

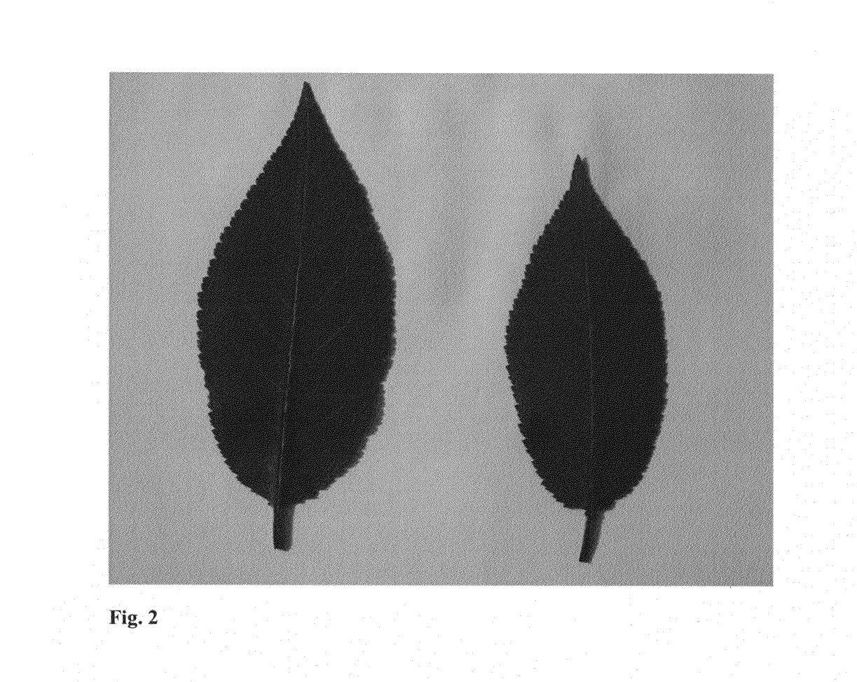

FIG. 3 provides a close-up view of fruits, branches and leaves of ‘STO2.’

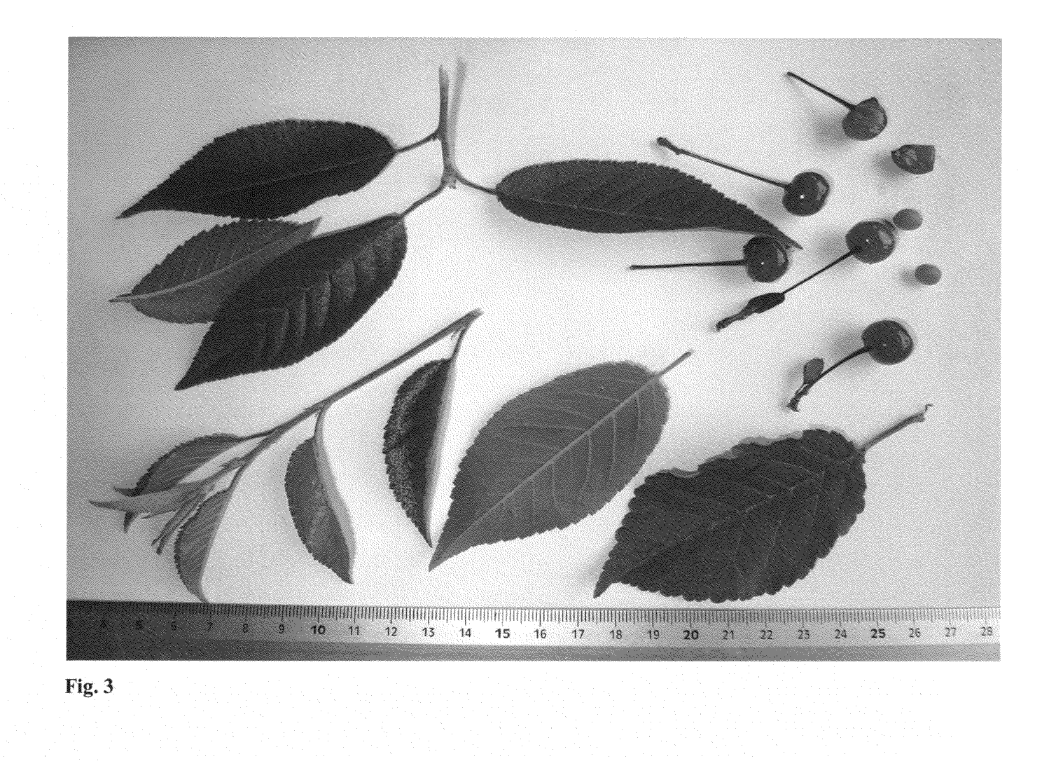

### DETAILED BOTANICAL DESCRIPTION

The aforementioned photographs, following observations and measurements describe plants grown during the summer in Kessbronn, Germany in an outdoor orchard and under conditions and practices generally used in commercial cherry production. Plants were 12 years old when the photographs and descriptions were taken. Measurements and numerical values represent averages for typical plants and plant parts. The actual measurements of any individual plant or plant parts, or any group of plants or plant parts, of the new Prunus plant may vary from the stated average. In the following description, color references are made to The Royal Horticultural Society (R.H.S.) Colour Chart, except where general terms of ordinary dictionary significance are used.

‘STO2’ has a generally arched plant habit and is weak in growth. The weeping plant habit is typical for the new Prunus plant. After twelve growing seasons in Kressbronn, Germany, a height of about 3.7 meters, a width of about 2.95 meters and a trunk diameter of about 9.3 cm were observed for ‘STO2.’ The growth reduction induced by ‘STO2’ allows a highly intensive cherry production with dwarf trees.
Botanical classification: Prunus cerasus L.×schmittii Rehder ‘STO2.’
Parentage: Breeding between Prunus cerasus and a Prunus canescens hybrid.
Propagation:
   Type.—By asexual reproduction.
   Method.—By tissue culture since the autumn of 2001 in a controlled environment. The process is similar to the propagation of other cherry rootstock.
Plant description:
   Plant use.—Only as a rootstock.
   Plant form.—Semi-ellipsoid form, after 3 years reflexed branching.
   Plant and growth habit.—Weeping plant habit; plant are typically grown as a single stem; weak vigor.
   Stem description.—Strength: Strong. Texture: Rough. Color: Grey brownish RHS 199B.
   Leaf description, fully developed leaves.—Arrangement: Alternate; simple. Length: About 8 cm to about 9.5 cm. Width: About 3.7 cm to about 4.5 cm. Shape: ovate, very elongated. Apex: Acute. Tip: Acuminate. Base: Obtuse, convex. Margin: Biserrate. Texture, upper surface: Glabrous. Texture, lower surface: Coarse, pubescent. Color: Fully developed leaves, Upper surface: Close to RHS 141B; Venation: Close to RHS 146C. Fully developed leaves, Lower surface: Close to144A; Venation, Close to RSH 144C.
   Petiole description.—Length: About 1.3 cm to about 1.9 cm. Diameter: About 0.2 cm. Color, upper surfaces and lower surfaces: Close to RHS161A.
   Presence of stipules.—Absent.
Flower description:
   Flower type/habit.—3 to 6 flowers single flowers arranged one compound panicles. Flowers occur in general on one year old branches.
   Fragrance.—Weak, pleasant.
   Natural flowering season.—In April, in south Germany, flowering occurs at the about the same time as ‘GI 2091,’ patented cherry rootstock (U.S. Plant Pat. No. 16,173).
   Lastingness of entire bloom period.—About 15 days.
   Lastingness of an individual bloom.—About 8 days.
   Inflorescence height.—About 3.5 cm to about 4 cm.
   Inflorescence diameter.—About 2.5 cm to about 3.5 cm.
   Flower diameter.—About 1.4 cm to about 1.8 cm.
   Flower depth.—About 0.8 cm to about 1.1 cm.
   Bloom quantity.—Fertile, a large quantity of flowers occur each year. Remarkably, the flowers occur on only the one year old branches.
   Petals.—Arrangement: Five petals in a single whorl. Slightly overlapping. Length: About 0.5 cm to about 0.9 cm. Width: About 0.5 cm to about 0.7 cm. Shape: Oblong, slightly elongated. Apex: Rounded, slightly elongated. Base: Truncate. Margin: Entire. Texture, upper and lower surfaces: Smooth, glabrous. Color: When fully opened, upper surface: Close to RHS 155B. When opening and fully opened, lower surface: Close to RHS 155B.
   Sepals.—Arrangement: Five sepals in a single whorl. Length: About 0.3 cm. Width: About 0.11 cm to about 0.23 cm. Shape: Angular set, triangular, slightly elongated. Apex: Pointed. Base: Truncate. Margin: Entire. Texture, upper and lower surfaces: Smooth, glabrous. Color, upper surfaces: Close to RHS 142B. Color, lower surfaces: Close to RHS 145B.
   Pedicels.—Length: About 1.5 cm to about 1.9 cm. Diameter: About 0.1 cm. Angle: About semi-erect to semi-prostrate from the inflorescence stalk. Texture: Smooth, glabrous. Color: Close to RHS 142A.
   Peduncle.—Length: About 0.2 cm to about 1.1 cm. Diameter: About 0.2 cm. Surface: Smooth. Color: Close to RHS 149A.
   Reproductive organs.—Stamens: Quantity per flower: Numerous. Filament length: About 4 mm to about 7 mm. Filament color: Close to RHS 155B. Anther length: About 0.3 mm to about 0.8 mm. Anther color: Close to RHS 6A. Pistils: Quantity per flower: One. Pistil length: About 8 mm to about 13 mm. Style length: About 6 mm to about 11 mm. Style color: Close to RHS 150C. Stigma shape: Round. Stigma color: Close to RHS 150A.
   Fruit description.—Maturity when described. Date picking: July in south Germany, varies with climatic conditions. About the same time as ‘GI 2091,’ patented cherry rootstock (U.S. Plant Pat. No. 16,173). Size: very small to small about 1 cm to about 1.3 cm diameter. Average weight: 1.2 g, varies slightly with fertility of the soil, amount of thinning and climatic conditions. Form: Globose, Slightly flat at the apex. Stem cavity: Slightly obcordate to flat. Color: RHS 185A.
   Peduncle description.—Size: Average length about 3.4 cm. Average diameter about 0.09 cm. Color: RHS 149A.
   Stone description.—Average length: About 0.74 cm. Average width: About 0.6 cm. Average thickness: 0.55 cm. Form: Nearly globose. Base: round. Apex: Rounded. Color: RHS 163C when dry.
   Fruit use.—not to be used as a dessert fruit.
Temperature tolerance: Plants of the new Prunus have been observed to tolerate temperatures from about −20° C. to about 40° C.

What is claimed is:

1. A new and distinct cultivar of Prunus cerasus L.×schmittii Rehder named ‘STO2,’ substantially as illustrated and described herein.
